(19) United States
(12) Patent Application Publication
(10) Pub. No.: US 2017/0143528 A1
(43) Pub. Date: May 25, 2017

(54) Methods and Devices for Correcting Pectus Carinatum

(71) Applicant: Joseph S. Pongratz, Chandler, AZ (US)

(72) Inventor: Joseph S. Pongratz, Chandler, AZ (US)

(21) Appl. No.: 15/358,041

(22) Filed: Nov. 21, 2016

Related U.S. Application Data
(60) Provisional application No. 62/257,297, filed on Nov. 19, 2015.

(51) Int. Cl. A61F 5/02; A44B 11/00; A41F 3/00

(52) U.S. Cl. CPC A61F 5/02; A41F 3/00; A44B 11/00

(57) ABSTRACT

An embodiment can include a pectus carinatum device for providing an adjustable compressive force to a subject's sternal protrusion. A pectus carinatum device can extend around a subject's torso, and chest cavity and apply a dynamic compressive pressure directly to a subject's sternal protrusion. A pectus carinatum device can include a strap removably attached to a clasp, a cinching device, a compression plate, and a protective padding. A cinching device and compression plate can apply a dynamic compressive force to a subject's sternal protrusion. A strap applies tension around a subject's torso, but allows the subject to freely move, and breath while keeping a constant dynamic compressive force on a subject's sternal protrusion. A protective padding can be removably attached to a compression plate, and an enclosure can be configured to surround a strap and to receive anti-migration, and antimicrobial material.

This application is a nonprovisional of U.S. provisional application No. 62/257,297, filed Nov. 19, 2015 with the same title, incorporated herein in its entirety.

### TECHNICAL FIELD

The present specification relates to a device and method for non-surgically correcting pectus carinatum.

### BACKGROUND

Various types of devices for treating pectus carinatum are known in the art. Usually pectus carinatum is treated by surgery, which can include excisions of the deformed cartilage with sternal osteotomy and an internal fixation device implanted into the subject. Surgically treating pectus carinatum often provides an individual with poor long term outcomes and a lengthy recover process. With surgery and implants, pectus carinatum could reoccur in a subject over time. As a result, non-surgical methods and devices were developed to treat pectus carinatum. Typically, a pectus carinatum device has a large front plate placed on a subject's chest such that the plate applies pressure to the pectus carinatum site. The large front plate is usually attached to a metal frame, which can cause skin ulcerations or back pain. The large front plate is usually custom made to fit the varying size and shape of each subject's chest. Furthermore, previous pectus carinatum devices usually require multiple configuration points to accommodate the subject's chest size and width of the subject's torso.

A pectus carinatum device uses Wolff s Law to move a subject's bone. Wolff s law states that when healthy bone and cartilage are being loaded with a constant force, the bone will adapt, strengthen, and eventually reform itself under the pressure. Typically, a pectus carinatum device pushes against a subject's bone with a constant force. Over time the force on the bone requires adjustment to fit the subject's pressure requirements. With current technologies, a pectus carinatum device can either be replaced with a new device or can require the subject to take the device to an expert to have it adjusted. Typically, a pectus carinatum device uses a large front plate with an opposing force from a large back plate placed on the back of the subject. In addition, metal frames around the subject's torso, along with straps around the shoulders, keep the front and back plate in position. This can cause the subject discomfort and/or significantly limit the subject's movement. Furthermore, subjects usually have to wear a pectus carinatum device for long periods of time to change the bone structure, which can eventually lead to bacteria growth on the device, and strong smelling odors, because current devices are not completely washable. There is a need for a pectus carinatum device that can be quickly adjustable and customizable to the subject's torso and force requirements for changing the subject's bone structure, that is antimicrobial, and that can allow the subject to freely move without being obstructed by the pectus carinatum device.

### SUMMARY

Aspects disclosed herein relate to a device for applying a pressure on a subject's chest, more specifically a pectus carinatum device for providing an adjustable compressive force to a sternal protrusion. A pectus carinatum device can include a strap that can be configured to wrap around and contour itself to a subject's torso and/or chest cavity. Sections of the torso and/or chest cavity can include one or more sections such as, for example, the body of sternum, sternal angle, manubrium sterni, and xiphoid process. In an exemplary embodiment a pectus carinatum device can wrap around one or more portions of a subject's anterior chest wall, sternum, anterior thorax, lateral thorax and posterior thorax regions. A strap can have a first end and a second end, and an exterior portion and an interior portion, joined at a top portion and a bottom portion. A strap can be one or more straps. A strap's first end can be removably connected to a first clasp. A first clasp can be configured to connect a strap and a cinching device together through an attachment receiver. A cinching device can be removably connected to a first clasp by an attachment receiver. A cinching device can be configured to accept one or more laces that can be removably attached to a first front clasp. A first front clasp can be removably connected to a front strap.

A cinching device can pull or release tension through one or more laces which can react on a compression plate, which can apply a set dynamic compressive pressure to a subject's sternal protrusion. A cinching device and compression plate combination can apply a set dynamic compressive pressure to a subject's sternal protrusion. In certain embodiments a compression plate can be interchangeable with another compression plate with a different dynamic compressive load, or different size to adjust to a subject's load pressure and/or compression requirements needed during treatments. In addition, a subject's sternal protrusion load pressure or compression requirements can be adjusted by adjusting the tension on a cinching device. A subject's load pressure or compression requirements can be measured in pounds per square inch by one or more or a combination of a cinching device, a compression plate, a load cell, a strain gage attached to a compression plate, or a fiber optic sensor.

In embodiments a compression plate can have an exterior and an interior surface with a first and second end. A compression plate's exterior side can be configured to removably accept an interior side of a front strap. A front strap can contour itself around the exterior surface of a compression plate, and can be removably connected to the compression plate's exterior surface. A compression plate can have a radius, a flat, or a point at its base and can gradually slope away from its radius base, flat base, or pointed base to its first and second ends. In embodiments a protective padding can be configured to be removably attached to a compression plate's interior surface. In certain embodiments, a protective padding can extend from a compression plate's first end to a compression plate's second end. A pectus carinatum device can include a cover that can wrap around a compression plate, and a protective padding, and extend from a compression plate's first end to a compression plate's second end. In an exemplary embodiment a cover can be removably attached to a compression plate, and a protective pad. In certain embodiments a cover can be permanently attached to a compression plate and a protective pad.

In embodiments a front strap's second end can be removably connected to a front second clasp, which can be removably connected to a second clasp. A second clasp can be configured to be removably attached to a second end of a strap. An enclosure can include one or more portions of an interior side and exterior side having a first end and a second end, where the interior side and exterior side can be joined at a top portion and a bottom portion forming an enclosure for a strap to slid through. A strap can be slide through an enclosure and an enclosure can cover some of or all of a strap. An enclosure can be antimicrobial, with silver lined material attached or weaved into an interior side and exterior side of the enclosure. An enclosure can have a thermal absorbing material and/or anti-migration material attached to its interior side at one or more portions.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 shows a top view of a pectus carinatum device, in accordance to one, or more embodiments;

FIG. 2 shows a top view of a pectus carinatum device with a cover and an enclosure not shown, in accordance to one, or more embodiments;

FIG. 3 shows a general side view of one embodiment of a cinching device of a pectus carinatum device;

FIG. 4 shows a general front view of a pectus carinatum device, in accordance to one, or more embodiments;

FIG. 5 shows an interior view of a pectus carinatum device with a cover not shown, in accordance to one, or more embodiments;

FIG. 6 shows a detailed view of a protection pad, compression plate, and front strap, in accordance to one, or more embodiments;

FIG. 7 shows one or more embodiments of a compression plate;

FIG. 8 shows a bottom view of a pectus carinatum device, in accordance to one, or more embodiments;

FIG. 9 shows an additional embodiment of a cinching device of a pectus carinatum device, in accordance to one, or more embodiments;

FIG. 10 shows a top view of an additional embodiment with a cinching device on both ends of a strap, in accordance to one, or more embodiments;

FIG. 11 shows an interior view of an additional embodiment with a cinching device on both ends of a strap, in accordance to one, or more embodiments; and

FIG. 12 illustrates a method for applying compression to a pectus region of a subject using a pectus carinatum device, in accordance with one or more embodiments.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

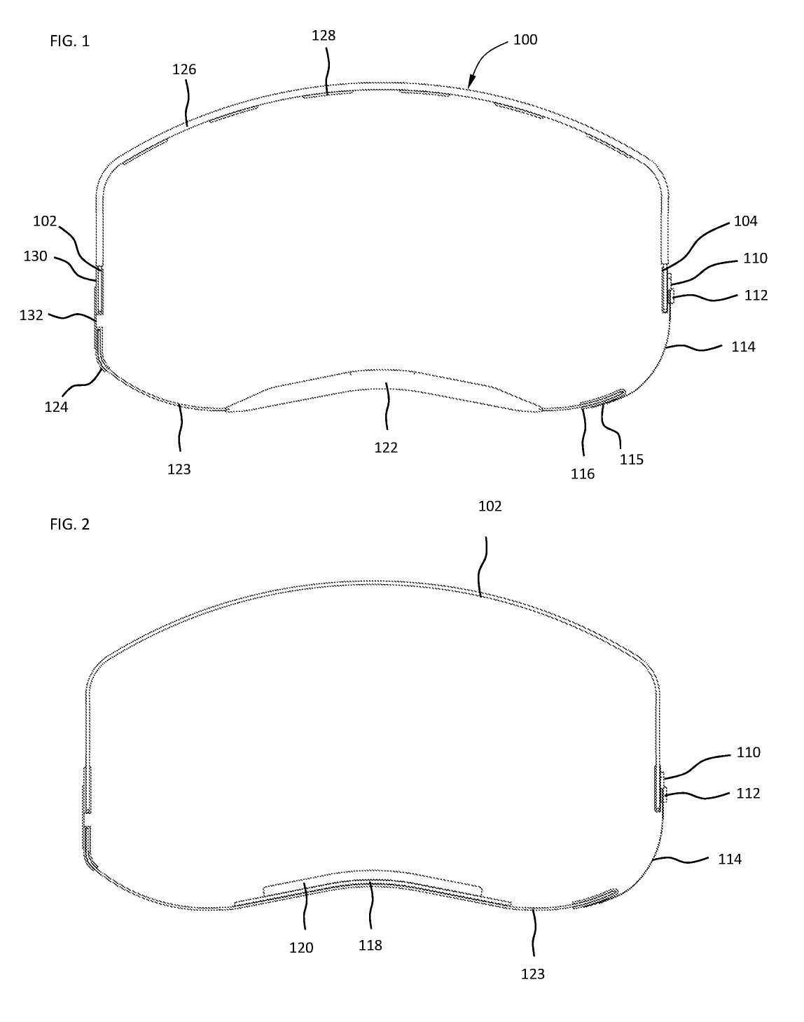

Referring initially to FIG. 1 and FIG. 2, shows a top view of a pectus carinatum device 100, and a top view of a pectus carinatum device with a cover 122 and an enclosure 126 removed for clarity, which can provide an external dynamic compressive force against a subject's sternal protrusion. In embodiments a subject's chest cavity can include one or more sections of such as, for example, the body of sternum, sternum, sternal angle, manubrium of sternum, and xiphoid process. A pectus carinatum device may extend over and/or apply pressure around a subject's thorax region, which can include one or more portions of a subject's anterior chest wall, sternum, anterior thorax, lateral thorax and posterior thorax regions. In embodiments a pectus carinatum device can apply a quantifiable dynamic pressure to a subject's sternal protrusion by gradually compressing the subject's sternum backwards continuously until it reshapes itself into normal position (Wolff's Law Principles). A pectus carinatum device can apply dynamic compressional force to a subject's sternal protrusion while allowing lateral thoracic expansion.

Embodiments include a strap 102 which can have a first end and a second end, and an exterior side and an interior side joined at a top portion and a bottom portion. A strap 102 can substantially wrap, contour, and/or extend itself around a subject's torso, and/or back, and/or chest cavity. A strap 102 can be such as, for example, 0.25 inches, 0.5 inches, 0.75 inches, 1 inch, 1.25 inches, 1.5 inches, 1.75 inches, 2 inches, 2.25 inches, 2.5 inches, 2.75 inches, 3 inches, or the like in height. A strap 102 can be such as, for example, 0.01 inches, 0.05 inches, 0.075 inches, 0.1 inch, 0.125 inches, 0.15 inches, 0.175 inches, 0.2 inches, 0.225 inches, 0.25 inches, 0.275 inches, 0.3 inches, or the like in width. A strap 102 can be adjustable in length, and can be cut and/or adjusted to fit a subject's chest size, and/or back size, and/or torso size from about 15 inches to about 50 inches. A strap 102 can be one or more straps such as, for example, one strap, two straps, three straps, four straps, five straps, six straps, seven straps, or the like.

In an exemplary embodiment a strap 102 can be dynamic such that it can stretch with a subject's body movement, and/or as a subject breathes in and out, a strap can still provide constant pressure and support to a subject's sternal protrusion thus not restricting respirations as a subject's respirations. In another embodiment a strap 102 can be static providing a constant and consistent pressure to a subject's torso and/or chest cavity, and a subject's sternal protrusion. A strap's 102 first end can be removably connected to, attached to, or can be fed through to a first clasp 104 such as, for example, an alligator strap, a metal loop, a flat metal slide, a rounded metal slide, a clasp buckle, a side release buckle, a cam buckle, a spring buckle, sewed onto a base for a tightening device, or the like. A strap's 102 second end can be removably connected to, attached to, or can be fed through to a second clasp 130 such as, for example, an alligator strap, metal loop, flat metal slide, rounded metal slide, clasp buckle, side release buckle, cam buckle, spring buckle, sewed onto a base for a tightening device, or the like. In embodiments a strap 102 can be such as, for example, a self-gripping strap, a hook and loop fastener strap, a flexible strap, nylon strap, elastic strap, flexibly resilient fabric, blend of nylon, spandex, polypropylene, cotton, polyester, elastic, or the like. In certain embodiments a strap 102 can be formed of one or more layers such as, for example, one layer, two layers, three layers, four layers, five layers, or the like.

In one embodiment a strap 102 can be removably connected to a clasp 104, which can be an alligator strap. A clasp 104 can form itself around a strap 102. A clasp 104 can have an attachment receiver 110 removably or permanently attached to it by such as, for example, 110 sewing it on, riveting it on, gluing it on, or the like. An attachment receiver 110 can be such as, for example, a click receiver, a cam buckle, a spring buckle, a clasp buckle, a receiver for a ratchet buckle, or the like. In another embodiment a strap 102 can be connected to itself by a hook and loop fastener through a metal loop, a flat metal slide, a rounded metal slide, or the like, wherein the metal loop can be connected to a clasp 104. In yet another embodiment a strap 102 can be connected to a clasp 104 by a clasp buckle, a side release buckle, a cam buckle, a spring buckle, or the like. A clasp 104 can have an attachment receiver 110 either removably attached or permanently attached to it by such as, for example, sewing it on, riveting it on, gluing it on, screwing it on, loop and hook fastener, or the like.

In embodiments a pectus carinatum device 100, can include a compression plate 118 having a first and a second end, and an interior surface and an exterior surface, and a cinching device 112. In embodiments a cinching device 112 can be removably attached to an attachment receiver 110 that can be adapted to receive the cinching device through a click receiver, a cam buckle, a spring buckle, a clasp buckle, a receiver for a ratchet buckle, or the like. A cinching device 112 can be such as, for example, a ratchet strap system, a tightening system with laces, Boa Closure Guide System, or the like. Embodiments can include a lace 114, which can be one or more laces. A cinching device 112 can be configured to include one or more laces 114 that can be removably connected directly to a first end of a compression plate 118, or to a lace receiver 115 that can be removably connected to the first end of a compression plate or can be removably connected to a front first clasp 116 by such as, for example, hook and loop fasteners, bolts and nuts, rivets, or the like. A front first clasp 116 can be a such as, for example, an alligator strap, a metal loop, a flat metal slide, a rounded metal slide, a clasp buckle, a side release buckle, a cam buckle, a spring buckle, sewed onto a base for a tightening device, or the like.

In embodiments a front strap 123 can have an inner surface and an outer surface having a first end and a second end, where the inner surface and outer surface can be joined at a top portion, and a bottom portion. A front first clasp 116 can be removably connected to a front strap 123 by such as, for example, hook and loop fastener, rivet, screws and bolts, or the like. A front first clasp 116 can be removably connected to a first end of a front strap 123. A front strap's 123 inner surface can contour itself to a compression plate's exterior surface 118, and removably attach itself by such as, for example, hook and loop fastener, rivet, screws and bolts, or the like. A front's strap 123 second end can be removably attached to a front second clasp 124 by such as, for example, hook and loop fastener, rivet, screws and bolts, or the like. A front strap 123 can be such as, for example, 0.25 inches, 0.5 inches, 0.75 inches, 1 inch, 1.25 inches, 1.5 inches, 1.75 inches, 2 inches, 2.25 inches, 2.5 inches, 2.75 inches, 3 inches, or the like in height. A front strap 123 can be such as, for example, 0.01 inches, 0.05 inches, 0.075 inches, 0.1 inch, 0.125 inches, 0.15 inches, 0.175 inches, 0.2 inches, 0.225 inches, 0.25 inches, 0.275 inches, 0.3 inches, or the like in width.

In embodiments a protective padding 120 can be removably attached onto a compression plate's 118 interior surface. A protective padding 120 can be configured to form to, or adjust to a subject's sternal protrusion and/or chest region. A protective padding 120, and compression plate 118 can be surrounded by and/or enclosed by a cover 122. In embodiments a cover 122 can be removable from a protective padding 120, and a compression plate 118. A cover 122 can have a front portion and a back portion joining at a top portion, and a bottom portion can be left open for a zipper 140, buttons, hook and loop fasteners, lace, or the like that can enclose the bottom portion for easy removal of the cover from a protective padding 120, compression plate 118, and front strap 123 as shown in FIG. 8. In certain embodiments a cover 122 can be permanently attached to a protective padding 120, a compression plate 122, and front strap 123. A cover 122 can be such as, for example, antimicrobial material, silver lined material, neoprene, thermal absorbing material, anti-migration material, or the like.

In embodiments a strap's 102 second end, second clasp 130 can be removably connected to a front strap's 123 second end's, front second clasp 124 by a strap connector 132. In one embodiment a strap connector 132 can be removably connected to a second clasp 130, and a front second clasp 124 by such as, for example, hook and loop fastener, rivet, bolts and nuts, or the like. In another embodiment a strap connector 132 can removably connect a front strap 123, and a strap 102 directly without any clasps on the strap itself by such as, for example, hook and loop fastener, rivet, bolts and nuts, or the like. In yet another, embodiment a front strap 123 can be connected to a strap 102 by metal loop, a flat metal slide, a rounded metal slide, a clasp buckle, a side release buckle, a cam buckle, a spring buckle, sewed onto a base for a tightening device, or the like.

In embodiments an enclosure 126 can have an interior portion, and an exterior portion including one or more edges, such as, for example, a top edge, a bottom edge, a first outer edge, a second outer edge, a first inner edge, and a second inner edge that can form an enclosure. An enclosure 126 can surround all or a portion of a strap 102 over the entire length or portions of the strap such as, for example, an enclosure can surround at least 1 inch, at least 2 inches, at least 3 inches, at least 4 inches, at least 5 inches, at least 10 inches, at least 15 inches, at least 20 inches, at least 25 inches, at least 30 inches, at least 35 inches, at least 40 inches, at least 45 inches, at least 50 inches, or the like. An enclosure 126 can surround all or a portion of a strap 102 over the entire length or portions of the straps such as, for example, an enclosure can surround at most 1 inch, at most 2 inches, at most 3 inches, at most 4 inches, at most 5 inches, at most 10 inches, at most 15 inches, at most 20 inches, at most 25 inches, at most 30 inches, at most 35 inches, at most 40 inches, at most 45 inches, at most 50 inches, or the like. In certain embodiments an enclosure 126 can include a bottom portion left open for a zipper, buttons, hook and loop fasteners, lace, or the like for easy removal of the enclosure.

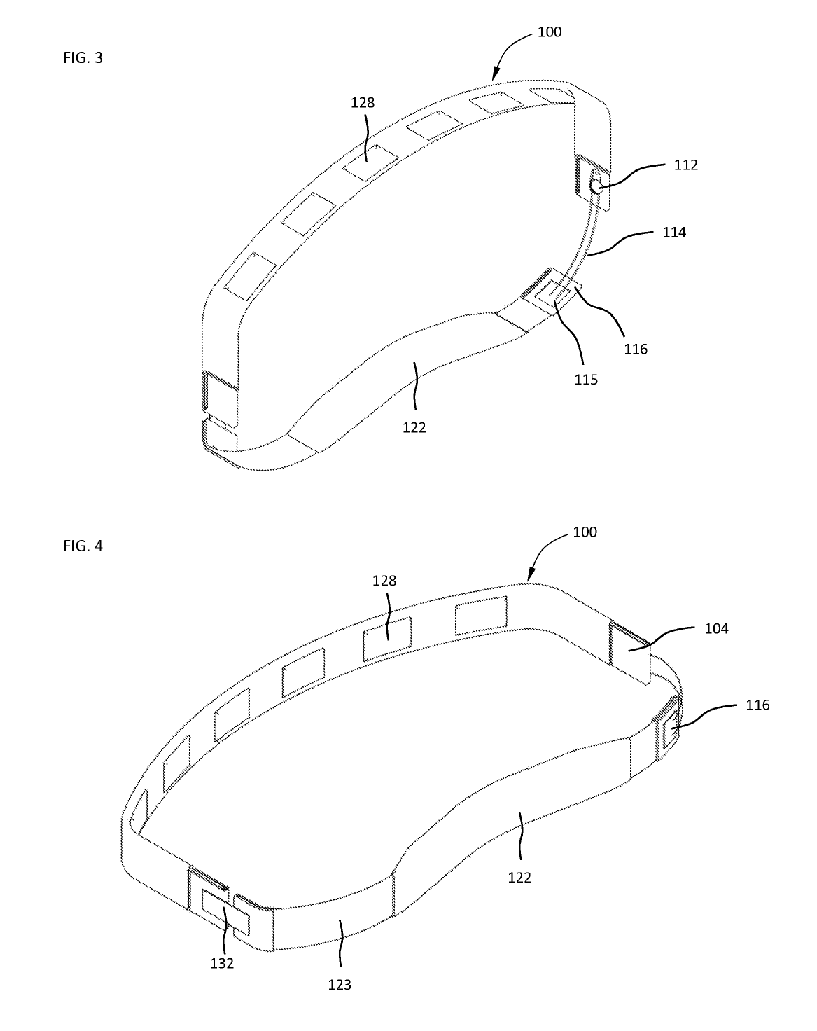

Referring to FIG. 3 and FIG. 4 shows a general side view of a cinching device, and a general front view of a pectus carinatum device, in accordance to one, or more embodiments. In embodiments a cinching device 112 can be configured to accept one or more laces 114. One or more laces 114 can be such as, for example, one lace, two laces, three laces, four laces, five laces, six laces, or the like. One or more laces 114 can be wire, polypropylene webbing, nylon webbing, metallic thread, wire thread, cord, or the like. One or more laces 114 can be removably attached to a compression plate 118 (not shown), or one or more laces can be configured to be removably attached to a lace receiver 116. A lace receiver 115 can be a click receiver with rivet or screw hole, nylon pad, plastic pad with rivet or screw holes, hook and loop fastener material, or the like. In certain embodiments a lace receiver 115 can be removably attached to a first end of a compression plate 118 (not shown). In another embodiment a lace receiver 116 can be removably attached to a front first clasp 116 by a such as, for example, loop and hook fasteners, bolts and nuts, rivets, or the like.

In embodiments a cinching device 112 through one or more laces 114 can apply or release lateral tension to a pectus carinatum device during a subject's treatment, which can apply a compressive force to a subject's sternal protrusion of at least 0.25 psi, at least 0.5 psi, at least 0.75 psi, at least 1 psi, at least 1.25 psi, at least 1.5 psi, at least 1.75 psi, at least 2 psi, at least 2.25 psi, at least 2.5 psi, at least 2.75 psi, at least 3 psi, at least 3.5 psi, at least 4 psi, at least 4.5 psi, at least 5 psi, at least 5.5 psi, at least 6 psi, at least 6.5 psi, at least 7 psi, at least 7.5 psi, at least 8 psi, at least 8.5 psi, at least 9 psi, at least 9.5 psi, at least 10 psi, or the like. A cinching device 112 can apply or release lateral tension to a pectus carinatum device during a subject's treatment which can apply a compressive force to a subject's sternal protrusion of at most 0.25 psi, at most 0.5 psi, at most 0.75 psi, at most 1 psi, at most 1.25 psi, at most 1.5 psi, at most 1.75 psi, at most 2 psi, at most 2.25 psi, at most 2.5 psi, at most 2.75 psi, at most 3 psi, at most 3.5 psi, at most 4 psi, at most 4.5 psi, at most 5 psi, at most 5.5 psi, at most 6 psi, at most 6.5 psi, at most 7 psi, at most 7.5 psi, at most 8 psi, at most 8.5 psi, at most 9 psi, at most 9.5 psi, at most 10 psi, or the like.

In embodiments lateral tension can be applied or released to a pectus carinatum device 100, and a compressive force can be applied or released to a subject's sternal protrusion in increments by a cinching device 112 through one or more laces 114 that can apply a compressive pressure in increments by a ratchet device or turning a closure system that clicks with each ratchet or turn, whereas one ratchet or click with a cinching device can apply or release 0.25 psi, or one ratchet or click with a cinching device can apply or release 0.5 psi, or one ratchet or click with a cinching device can apply or release 1 psi, or one ratchet or click with a cinching device can apply or release 1.25 psi, or one ratchet or click with a cinching device can apply or release 1.5 psi, or one ratchet or click with a cinching device can apply or release 2 psi, or one ratchet or click with a cinching device can apply or release 2.5 psi, or one ratchet or click with a cinching device can apply or release 3 psi, or one ratchet or click with a cinching device can apply or release 3.5 psi, or one ratchet or click with a cinching device can apply or release 4 psi, or one ratchet or click with a cinching device can apply or release 4.5 psi, or the like.

In certain embodiments a strap 102 can have an elastic tensioning force that can be greater than the compressive force of a compression plate 118 that can give sufficient compression on a compression plate, which can allow a strap to stretch with a subject's body movement and/or breathing without releasing the compression plate's compressive force against the subject's sternal protrusion.

In embodiments an enclosure 126 can be configured to have anti-migration patches 128 attached to it which can be textured such as, for example, dots, lines, waves, or the like that protrude from the surface of the material that can allow blood to flow more easily around a pectus carinatum device 100 and through the subject's back. In certain embodiments an anti-migration patch can be silicon beads, Setex™, anti-slip material, or the like. In certain embodiments anti-migration patch can be configured to include a protective foam underneath the patch to provide a subject's back and sides with a foam barrier between a strap 102 and a anti-migration patch 128. A protective foam can be plastazote, rubber, neoprene, polyethylene foam, polypropylene foam, latex foam, polyurethane foam, memory foam, sorbothane foam, silicone, silicone sheets, urethane, viscos material, closed or open cell foams or the like. A anti-migration patch 128 can be substantially spaced along an interior portion of an enclosure 126. In certain embodiments an anti-migration patch 128 can align the entire interior portion of an enclosure 126. In other embodiments an anti-migration patch 128 can be evenly spaced in an interior portion of an enclosure 126.

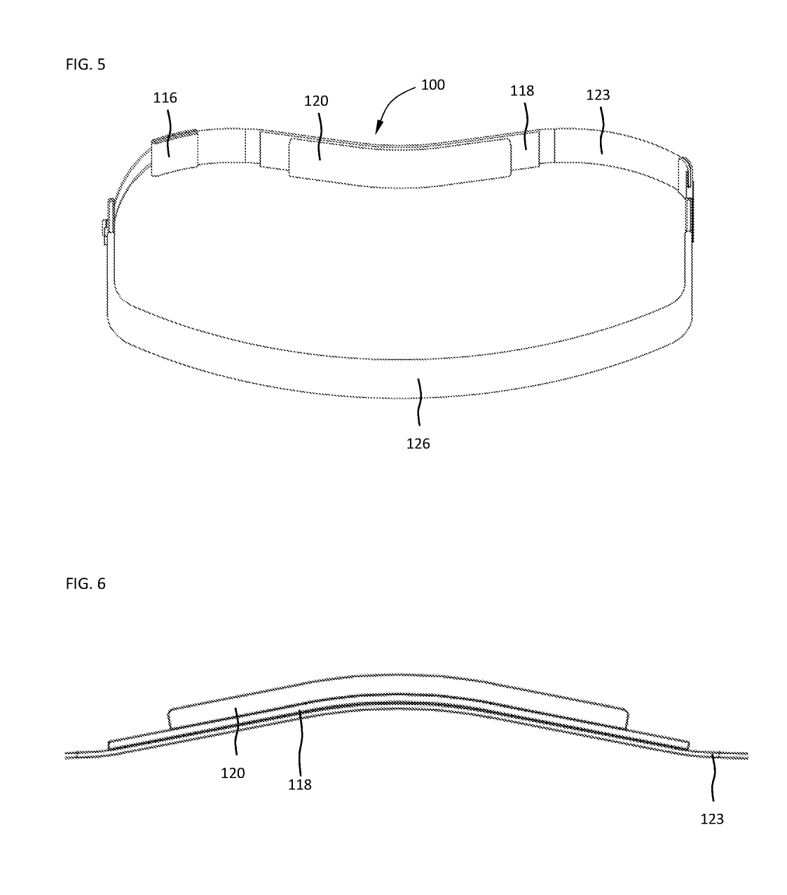

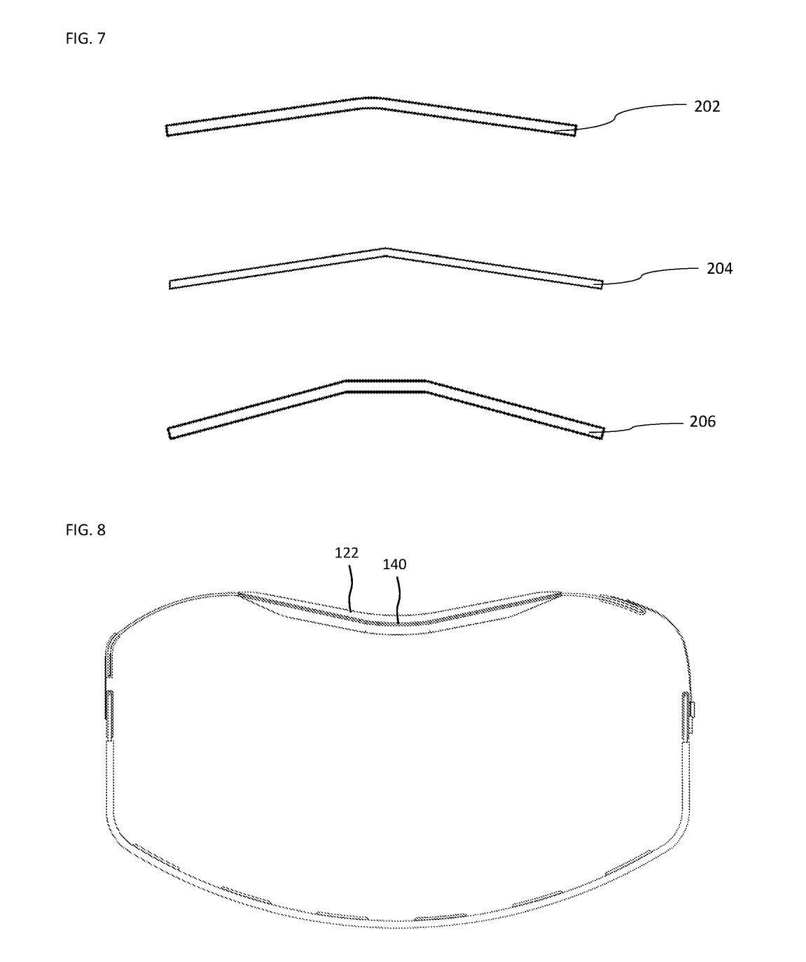

Referring to FIG. 5, FIG. 6, and FIG. 7, shows an interior view with a cover 122 not shown, a detailed view of a protection pad 120, compression plate 118, and front strap 123, and one or more embodiments of a compression plate in accordance to one, or more embodiments. In embodiments a compression plate 118 can have a first end and a second end, and an interior and an exterior side. A compression plate's 118 interior surface can be a side that is closest to a subject's sternal protrusion. An exterior side can be the opposite side furthest from the subject's sternal protrusion. In one embodiment a dynamic compression plate 202 can have a first and a second end wherein the first end can gradually slope into a radius forming an arc sweeping back into the second end. In another embodiment a compression plate 118 can have a first and a second end wherein the first and the second end gradually slope into a vertex as shown at 204. In yet another embodiment a dynamic compression plate can have a first and a second end wherein the first and second end gradually slopes into a flat surface as shown at 206. A compression plate 118 is not limited to the embodiments shown in FIG. 7. A compression plate 118 can be formable, and customizable to fit any shape of a subject's sternal protrusion. A compression plate 118 can perform similar to a spring such that when tension is applied to a pectus carinatum device 100 the compression plate can be compressed against a subject's sternal protrusion, and when the tension is released a compression plate can return to its original shape.

In certain embodiments a compression plate 118 can be replaced with another compression plates with different material properties, and/or spring constant, and/or, modulus of elasticity to adjust to a subject's changing chest and/or torso growth, and/or load compression and/or pressure requirements. In certain embodiments an additional compression plate 118 can be added to a compression plate 118 to add a compressional force to a subject's sternal protrusion. In certain embodiments a compression plate 118 can be an adjustable spring such as, for example, a leaf spring, a compression spring, a coil spring, a wave spring, a torsion spring, a wire form spring, a tension spring, polypropylene formed spring, thermoformed material, flat plastics, carbon graphite, Lexeon, or the like. In certain embodiments, a pectus carinatum device 100 can include one or more compression plates 118 such as, for example, one plate, two plates, three plates, four plates, five plates, or the like. A compression plate 118 can be formed of a variety of materials such as, for example, metals, plastics, polymers, rubbers, carbon graphite, flexone, Lexeon, steels, or the like. In certain embodiments a compression plate 118 can creep over time, which can give a subject's sternal protrusion less compressional force. A compression plate 118 can resist creep by adding anti-creep material within the compression plate such as stiffeners, and/or adding and/or aligning material such as metal, plastics, and steels to support a compression plate.

In certain embodiments a compression plate 118 and protective padding can be formed and/or molded to fit a subject's sternal protrusion. In some embodiments a subject's carinatum can be so severe that a standard compression plate will not fit the subject's sternal protrusion, so a custom compression plate 118 and protective pad 120 can be formed and/or molded to fit the subject's sternal protrusion. In certain embodiments measurements can be taken of a subject's sternal protrusions and a custom compression plate 118 and protective pad 120 can formed and/or molded from the subject's sternal measurements.

In embodiments when tension is applied or released to a pectus carinatum device 100 a compressive force can be applied or released to a compression plate 118 that can be set in increments such as, for example, at least 0.25 psi, at least 0.5 psi, at least 0.75 psi, at least 1 psi, at least 1.25 psi, at least 1.5 psi, at least 1.75 psi, at least 2 psi, at least 2.25 psi, at least 2.5 psi, at least 2.75 psi, at least 3 psi, at least 3.5 psi, at least 4 psi, at least 4.5 psi, at least 5 psi, at least 5.5 psi, at least 6 psi, at least 6.5 psi, at least 7 psi, at least 7.5 psi, at least 8 psi, at least 8.5 psi, at least 9 psi, at least 9.5 psi, at least 10 psi, at least 11 psi, at least 12 psi, at least 13 psi, at least 14 psi, at least 15 psi, at least 16 psi, at least 17 psi, at least 18 psi, at least 19 psi, or the like.

In embodiments tension can be applied or released to a pectus carinatum device 100 a compressive force can be applied or released to a compression plate 118 that can be set in increments such as, for example, at most 0.25 psi, at most 0.5 psi, at most 0.75 psi, at most 1 psi, at most 1.25 psi, at most 1.5 psi, at most 1.75 psi, at most 2 psi, at most 2.25 psi, at most 2.5 psi, at most 2.75 psi, at most 3 psi, at most 3.5 psi, at most 4 psi, at most 4.5 psi, at most 5 psi, at most 5.5 psi, at most 6 psi, at most 6.5 psi, at most 7 psi, at most 7.5 psi, at most 8 psi, at most 8.5 psi, at most 9 psi, at most 9.5 psi, at most 10 psi, at most 11 psi, at most 12 psi, at most 13 psi, at most 14 psi, at most 15 psi, at most 16 psi, at most 17 psi, at most 18 psi, at most 19 psi, or the like. A compressive force can be set by a compression plate 118 by its modulus of elasticity, the angle of a first and a second end, and the amount of distance that can be displaced when the compression plate is compressed when tension is applied or released. In certain embodiments a strain gage, a load cell, a fiber optic sensor, or the like can be removably attached onto a compression plate's interior or exterior surface. A strain gage, load cell, fiber optic sensor, or the like may be configured to, and/or removably attached to a compression plate to measure a compressive load seen by a subject's sternal protrusion. In certain embodiments a compression plate can be a spring, and Hooke's law can be applied to set a compressive force.

In embodiments a compression plate 118 can be configured to have protective padding 120 removably attached to it. A protective padding 120 can be removably attached to a compression plate 118 by such as, for example, hook and loop fastener, bolt and nut, hot glue, or the like. Protective padding can be formed of a variety of materials such as, for example, plastic, plastazote, rubber, neoprene, polyethylene foam, polypropylene foam, latex foam, polyurethane foam, memory foam, sorbothane foam, silicone, urethane, open or closed cell foams, viscous materials, silicone sheets, or the like. A protective padding 120 can form itself to a subject's sternal protrusion, and may be configured to extend across a compression plate's interior surface. In certain embodiments a protective padding's 120 exterior surface can be textured, or can have raised dots, raised lines, raised waves, raised squiggles, or the like to allow for blood to flow more easily through the subject's pressure area that can be created by a compression plate 118 and protective padding. In certain embodiments a protective padding 120 can be an air bladder, or a gel bladder.

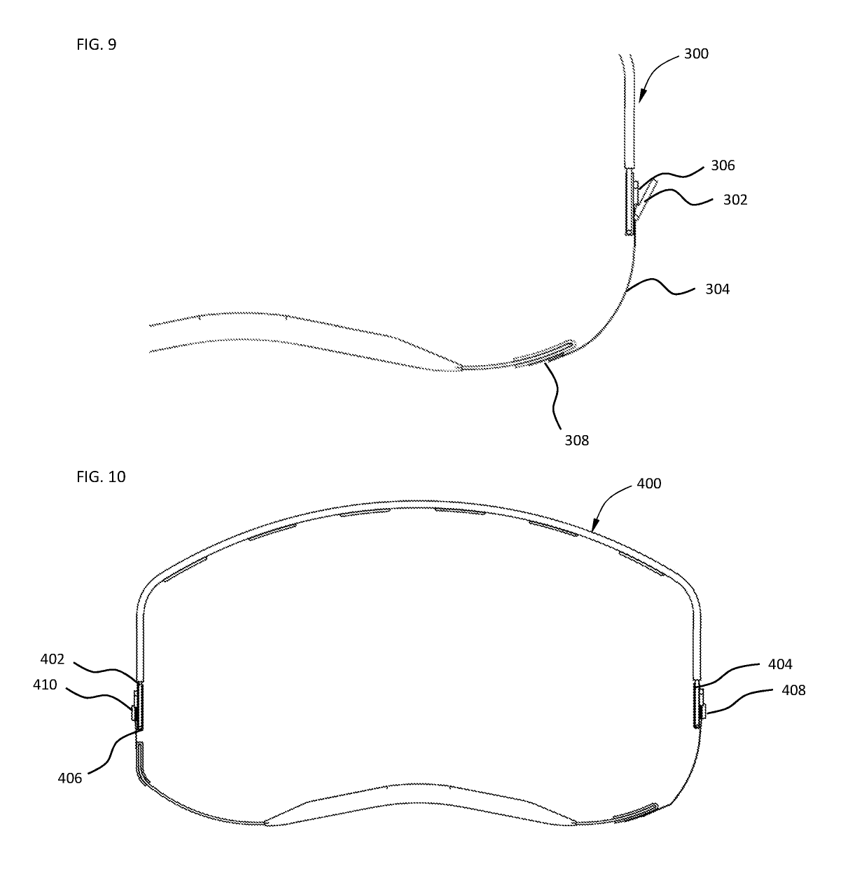

Referring to FIG. 9, shows an additional embodiment of a cinching device of a pectus carinatum device, in accordance to one, or more embodiments. A cinching device 302 can be a ratchet strap system. In certain embodiments a webbing strap 304 can be removably connected to a ratchet 302. A ratchet 302 can be removably connected to a ratchet connector 306. A ratchet 302, when not in use, can sit flush with a ratchet connector 306 so as to not irritate or get in the way of a subject's movement. A webbing strap 304 can be removably attached to a front clasp 308.

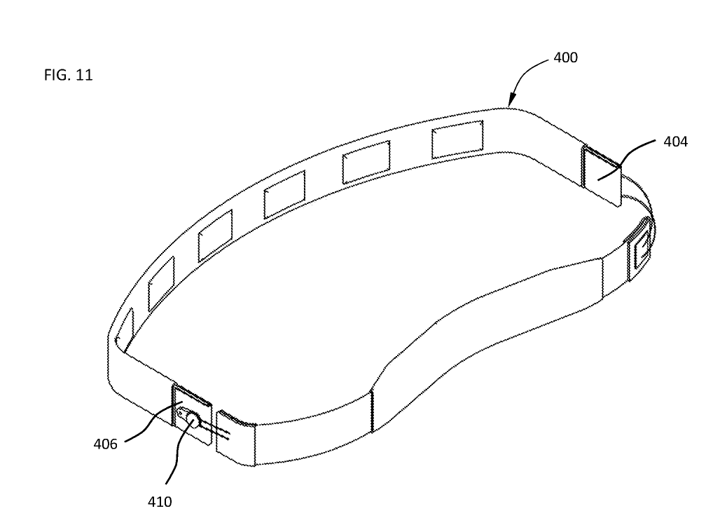

Referring to FIG. 10 and FIG. 11, shows a top view of an additional embodiment with a cinching device on both ends of a strap, and an interior view of an additional embodiment with a cinching device on both ends of a strap, in accordance to one, or more embodiments. In another embodiment of a pectus carinatum device 400 can have two cinching devices 408, 410 configured to be removably attached to both ends of a strap 402 by a first clasp 404 and a second clasp 406 removably attached to a first and second end of a strap.

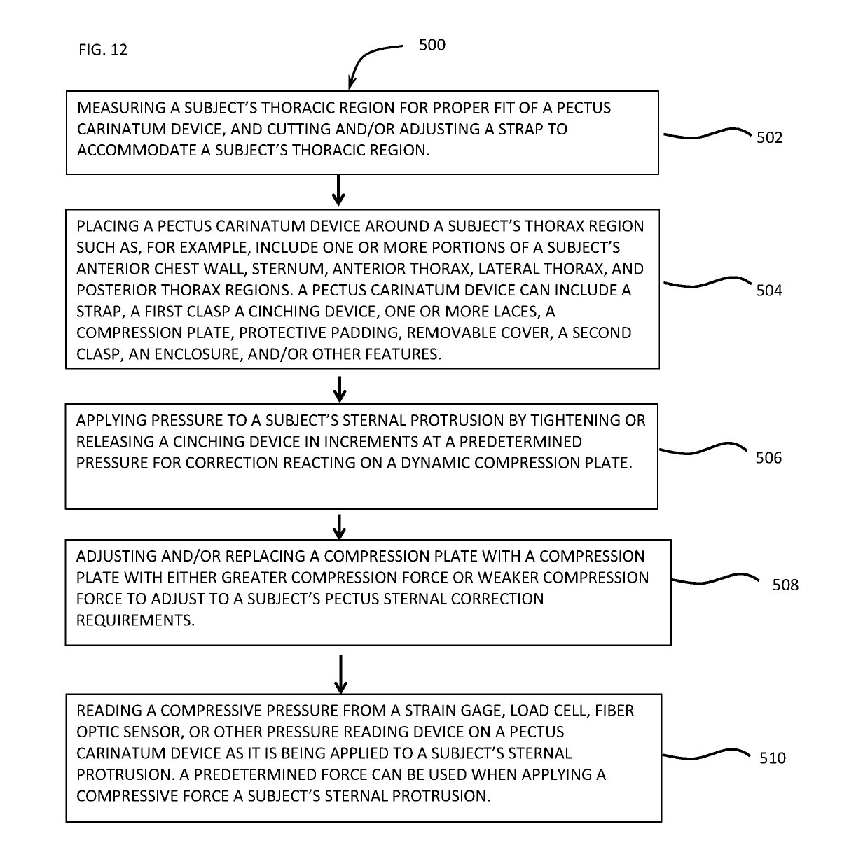

Referring to FIG. 12, illustrates a method 500 for applying compression to a pectus region of a subject using a pectus carinatum device. The operations of method 500 presented herein are intended to be illustrative. In certain embodiments, a method 500 can be accomplished by one or more additional operations not described, and/or in which the operations of method 500 are illustrated in FIG. 12 and described herein is not intended to be limiting.

At step 502, measuring a subject's thoracic region for proper fit of a pectus carinatum device, and cutting and/or adjusting a strap to accommodate the subject's thoracic region.

At step 504, placing a pectus carinatum device around a subject's thorax region, which can be such as, for example, include one or more portions of a subject's anterior chest wall, sternum, anterior thorax, lateral thorax and posterior thorax regions. A pectus carinatum device can include a strap, front strap, a first clasp, a cinching device, one or more laces, a compression plate, protective padding, removable cover, a second clasp, an enclosure, and/or other features.

A step 506, applying pressure to a subject's sternal protrusion by tightening or releasing a cinching device in increments. A predetermine pressure for correction can be applied to a subject's sternal protrusion by tightening or releasing a cinching device which can react on a compression plate keeping a dynamic compressive force on a subject's sternal protrusion.

At step 508, adjusting and/or replacing a compression plate can be applied to a pectus carinatum device. Step 508 can be performed with a compression plate that can apply such as, for example a compressive force that is greater than the force being applied, or a compressive force that is less than the force being applied to adjust to a subject's pectus sternal correction requirements.

At step 510, reading a compressive pressure from a strain gage, load cell, fiber optic gage, or other pressure reading device on a pectus carinatum device that is applied to subject's sternal protrusion in accordance to some embodiments. In some embodiments, step 510 can be performed as a pectus carinatum device is being applied cinched around a subject's sternal protrusion. A predetermined compressive force for a subject's sternal protrusion can be used and read with a strain gage, load cell, fiber optic gage, or other pressure reading device as the compressive force is being applied.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosed embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the embodiments otherwise claimed. No language in the present specification should be construed as indicating any non-claimed element essential to the practice of the disclosed embodiments.

### EXAMPLES

The following non-limiting examples are provided for illustrative purposes only in order to facilitate a more complete understanding of representative embodiments. These examples should not be construed to limit any of the embodiments described in the present specification including those pertaining to the method of correcting pectus carinatum.

### Example 1

### Correcting A Subjects Sternal Protrusion

A child has pectus carinatum characterized by an abnormal overgrowth of the coastal cartilages, which can result in a sternal protrusion, and adjacent costal cartilages. Using Wolff s law, pressure can be place on the child's sternum gradually forming and shaping it into normal position. A pectus carinatum device can be fitted to a child with a pectus carinatum. A pectus carinatum device is place around a child and the pectus carinatum device is adjusted to fit the child's torso and chest size through the strap, and compression plate. A set pressure is determined for the child's sternal correction. The correct compression plate for the determined pressure is inserted into the pectus carinatum device, and the pectus carinatum device is placed onto the child. The pectus carinatum device is then tightened with the cinching device applying the necessary compressive force to the compression plate, which reacts on the child's sternal protrusion. Over time the pressure will need to be adjusted which can be done with the cinching device or the compression plate. The pectus carinatum device allows a dynamic compressive force against a sternal protrusion while allowing the child to move freely, play sports, and have unrestricted breathing. In addition, the pectus carinatum device is antimicrobial decreasing bacterial growth and reducing odor and each part is removable and washable in a normal cycle washing machine.

What is claimed is:

1. A pectus carinatum device configured to provide adjustable dynamic pressure to a sternal protrusion of a subject, comprising:
a strap having one or more sections, having an exterior portion and an interior portion, joined at a top edge and a bottom edge, and a first end and a second end, said first end and said second end extends around sections of a subject's torso;
a first clasp is removably connected to said first end of said strap; an attachment receiver is removably connected to said first clasp; said attachment receiver is removably connected to a cinching device; said cinching device is configured to receive one or more laces; a compression plate having a first end and a second end, and an outer surface and an interior surface;
a front strap having one or more sections, having an exterior portion and an interior portion, joined at a top edge and a bottom edge, and a first end and a second end;
a front first clasp configured to be removably attached to said front strap's said first end; said one or more laces configured to be removably connected to said front first clasp; said front strap forming and removably attaching itself along said outer surface of said compression plate;
a protective padding removably attached to said interior surface of said compression plate; said compression plate and said protective padding is removably enclosed by a cover; a second clasp is removably connected to said second end of said front strap; and
an enclosure having an interior side, and an exterior side, and a top portion and a bottom portion where said interior side and said exterior side join at said top portion and said bottom portion forming an enclosure for said strap to slide through.

2. A pectus carinatum device of claim 1, wherein said protective padding is textured.

3. A pectus carinatum device of claim 1, wherein said compression plate and said cinching device is adjustable in compressive force from about 0.5 psi to about 30 psi.

4. A pectus carinatum device of claim 1, wherein said enclosure has anti-migration padding attached to it.

5. A pectus carinatum device of claim 4, wherein said anti-migration padding is textured.

6. A pectus carinatum device of claim 2, wherein said protective padding forms to a subject's sternal protrusion.

7. A method for for applying compression to a sternal protrusion of a subject using the pectus carinatum device of claim 1 comprising:
Measuring a subject's thoracic region, and cutting, and/or adjusting a strap to a fit a subject's thoracic region;
placing the pectus carinatum device around a subject's thorax regions including an anterior chest wall, a sternum, a anterior thorax, a lateral thorax, and a posterior thorax region via the strap, and removably connected to the compression plate by the first and the second clasp, and a cinching device;
applying pressure to a subject's sternal protrusion by tightening or releasing said cinching device in increments at a predetermined pressure for a correction force to adjust to a subject's pectus sternal requirements;
adjusting and/or replacing the compression plate to provide either a greater compression force or a weaker compression force to adjust to a subject's pectus sternal correction requirements; and
reading a compressive pressure from a strain gage, load cell, fiber optic sensor, or other pressure reading device as a pectus carinatum device is being applied to a subject's sternal protrusion.

8. A pectus carinatum device, for the treatment of a sternal protrusion, comprising:
a compression plate having a first end and a second end;
a strap assembly, for applying an applied tension to the compression plate, that positions at least a portion of the compression plate over at least a portion of the sternal protrusion;
an original shape characterized by an angle that the first end and the second end make when the applied tension is released; and
a dynamic compressive force when the applied tension forces the compression plate against a sternal protrusion enough to change the angle of the compression plate.

9. The pectus carinatum device of claim 8 wherein a central portion of the compression plate is characterized by a bend that substantially defines the angle of the original shape.

10. The pectus carinatum device of claim 8 wherein a central portion of the compression plate is characterized by a gradual arc that substantially defines the angle of the original shape.

11. The pectus carinatum device of claim 8 wherein a central portion of the compression plate is characterized by a substantially flat portion on either side of bends, and wherein the sum of angles of the bends substantially defines the angle of the original shape.

12. The pectus carinatum device of claim 8 wherein the strap assembly is frameless.

13. The pectus carinatum device of claim 8 wherein the strap assembly is characterized by a cinching device that is used to adjust a displaced distance of the first end and the second end, relative to the original shape, such that a desired dynamic compressive force is applied.

14. The pectus carinatum device of claim 8 further comprising a protective padding fixed to at least a portion of the compression plate.

15. The pectus carinatum device of claim 8 wherein the compression plate is a material having a modulus of elasticity and/or spring constant that provides a dynamic compressive force between 0.5 psi and 30 psi.

16. The pectus carinatum device of claim 8 wherein the dynamic compressive force is substantially consistent during normal respiration of a subject, even when there is a change in the angle of the compression plate, provided the compression plate does not return to the original shape during respiration.

17. A method for using the pectus carinatum device of claim 8 comprising the steps of: positioning a central portion of the compression plate against a sternal protrusion such that the first end and the second end are spaced away from a subject's thoracic region; and adjusting the strap assembly such that the dynamic compressive force is always present during respiration.

18. The method of claim 17 further comprising the step of cinching the strap assembly such that the first end and second end are pulled closer to the subject's thoracic region.

19. The method of claim 17 further comprising the step of positioning a protective padding between the compression plate and the sternal protrusion.

20. The method of claim 17 further comprising the step of trimming the compression plate to reduce the dynamic compressive force.
